(19) United States
(12) Patent Application Publication
(10) Pub. No.: US 2023/0282340 A1
(43) Pub. Date: Sep. 7, 2023

(54) DATABASE MANAGEMENT SYSTEM FOR DYNAMIC POPULATION STRATIFICATION BASED ON DATA STRUCTURES HAVING FIELDS STRUCTURING DATA RELATED TO CHANGING ENTITY ATTRIBUTES

(71) Applicant: ODH, Inc., Princeton, NJ (US)

(72) Inventor: Adam JOHNSON, Princeton, NJ (US)

(73) Assignee: ODH, Inc., Princeton, NJ (US)

(21) Appl. No.: 18/115,487

(22) Filed: Feb. 28, 2023

Related U.S. Application Data
(63) Continuation of application No. 16/379,776, filed on Apr. 9, 2019, now abandoned.
(60) Provisional application No. 62/655,160, filed on Apr. 9, 2018.

(51) Int. Cl. G16H 40/20; G16H 10/60; G06F 16/2455; G06F 16/28; G16H 50/30

(52) U.S. Cl. CPC G16H 40/20; G16H 10/60; G06F 16/24564; G06F 16/288; G06F 16/285; G16H 50/30

(57) ABSTRACT

Methods, systems, and apparatus, including computer programs encoded on a storage device, for dynamic population stratification based on changing entity attributes. In one aspect, a method includes actions of obtaining data from a data source that describes attributes associated with an entity, determining, based on the obtained data, a first ranking of the entity based on the severity state of the entity, determining, based on the obtained data, a second ranking of the entity based on the acuity state of the entity, adjusting the first ranking of the entity based on the second ranking of the entity, and assigning the entity to a particular risk category based on the adjusted ranking.

### CROSS-REFERENCE TO RELATED APPLICATIONS

This application is a continuation of U.S. patent application Ser. No. 16/379,776; filed Apr. 9, 2019, which claims the benefit of the U.S. Provisional Patent Application No. 62/655,160 filed Apr. 9, 2018, and entitled “Search, Retrieval, and Classification of Population Stratification Data,” which is incorporated herein by reference in its entirety.

### BACKGROUND

Different entities may each be associated with different categories of risk and cost.

### SUMMARY

According to one innovative aspect of the present disclosure a method performed by a data processing system for dynamic population stratification based on data structures having fields structuring data related to changing entity attributes. In one aspect, the method may include actions of methods that include obtaining data from one or more data sources that describes attributes associated with a plurality of entities, determining, based on the obtained data, a first ranking of the plurality of entities, wherein the first ranking is based on a first measure of the state of each respective entity, determining, based on the obtained data, a second ranking of the plurality of entities, wherein the second ranking is based on a second measure of the state of each respective entity, adjusting the first ranking of the plurality of entities based on the second ranking of the plurality of entities, and clustering each of the plurality of entities to a particular risk category based on the adjusted rankings.

Other versions include corresponding systems, apparatus, and computer programs to perform the actions of method defined by instructions encoded on computer storage devices.

These and other versions may optionally include one or more of the following features. For instance, in some implementations, determining, based on the obtained data, a first ranking of the plurality of entities comprises ranking the plurality of entities based on the severity of the state of each respective entity.

In some implementations, determining, based on the obtained data, a second ranking of the plurality of entities comprises ranking the plurality of entities based on the acuity of the state of each respective entity.

In some implementations, ranking the plurality of entities based on the acuity of the state of each respective entity comprises evaluating a plurality of factors that includes one or more of (i) a recent trend score, (ii) an experience-based score, and (iii) an impact score.

In some implementations, method can further include determining the recent trend score for each particular entity of the plurality of entities, wherein determining the recent trend score for the particular entity is based on a comparison of a recent cost associated with the particular entity and a long-term cost associated with the particular entity.

In some implementations, the method can further include determining the experience-based score for each particular entity of the plurality of entities, wherein determining the experience-based score for the particular entity is based on data indicating the particular entity's utilization of a plurality of different health care services.

In some implementations, determining the experience-based score for the particular entity based on data indicating the particular entity's utilization of a plurality of different services can include obtaining a set of rules that are related to the use of each respective service of the plurality of different services, wherein each rule of the set of rules establishes a threshold number of uses for each service of the plurality of different services within a particular time period, determining, for each particular service of the plurality of different services, a number of instances that the particular entity availed itself of the particular service more than the threshold number of times with the particular time period, aggregating the number of instances that the particular entity availed itself of each service of the plurality of services more than the threshold number of times within the particular time period, and determining an experience-based score for the particular entity based on the aggregated number of instances.

In some implementations, the method can further include determining the impact score for each particular entity of the plurality of entities, wherein determining the impact score for each particular entity is based on the particular entity's historical pathway markers, present care pathway markers, or a combination of historical pathway markers and present pathway markers.

These and other innovative aspects of the present disclosure are escribed more detail below, in the accompanying figures, and in the claims.

### BRIEF DESCRIPTION OF THE DRAWINGS

FIG. 1 is a contextual diagram of an example of a database management system for dynamic population stratification based on data structures having fields structuring data related to changing entity attributes.

FIG. 2 is a flowchart of an example of a process for dynamic population stratification based on changing entity attributes.

FIG. 3 is another flowchart of another example of a process for dynamic population stratification based on changing entity attributes.

FIG. 4 is a block diagram of system components that can be used to implement a database management system for dynamic population stratification based on data structures having fields structuring data related to changing entity attributes.

### DETAILED DESCRIPTION

Innovative aspects of the present disclosure identify strategies for reducing entity risk by first clustering entity data structures, that each have fields structuring data describing one or more attributes of an entity, into an initial population tier and then applying one or more rules to each clustered entity data structure. Each rule may include a rule data structure that includes fields representing programmed logic that, when executed by one or more computers, performs a series of one or more operations on attributes of the entity structured by the entity data structure. Application of the one or more rules to the clustered entity data structures by an entity tiering algorithm provides an indication of one or more strategies that can be used to move one or more of the entities represented one of the clustered entity data structures to a different population tier that is associated with less risk.

Aspects of the present disclosure are directed towards a population tiering algorithm that can be used to determine an initial population tier for a single entity data structure. The entity data structure may be generated based on input received from a user of a user device identifying an entity. Alternatively, aspects of the present disclosure can execute the entity tiering algorithm described by the present disclosure to assign each entity data structure in one or more databases into a respective population tier.

The present disclosure provides an entity population tiering algorithm that can analyze a number of entity attributes structured by fields of an entity data structure. In some implementations, the entity attributes may include data describing services used by the entity, programmatic data describing programs participated in by the entity, and social data such as social media data related to the entity to cluster a population of entities represented by multiple entity data structures in one or more database into an initial tier of a plurality of tiers based on severity and acuity of each respective entity's medical condition.

The present disclosure can identify one or more entity data structures as candidate entities that have potential for moving to a different tier that is associated with less risk. The application of one or more rules to the entity data structures can identify one or more strategic programs that the corresponding entity can participate in to help reduce the entities risk based on an analysis of one or more factors that can include impactability and intensity of intervention. In some implementations, identifying one or more strategic programs can include sorting entity data structures representing respective entities into buckets of entity data structures associated with one or more programs. Such programs may include case management programs, care coordination programs, disease management programs, or the like. Buckets may include, for example, electronic directories that are tagged with a program identifier and represent a storage location where entity data structures for entities who are candidates for a program identified by the directory can be stored. The entity population tiering algorithm can be periodically repeated weekly, monthly, annually, or the like to ensure timely intervention for entities needing intervention. Likewise, periodic execution of the tiering algorithm can be performed to facilitate recognition of entities for whom effective intervention has lowered their acuity, severity, or overall risk levels.

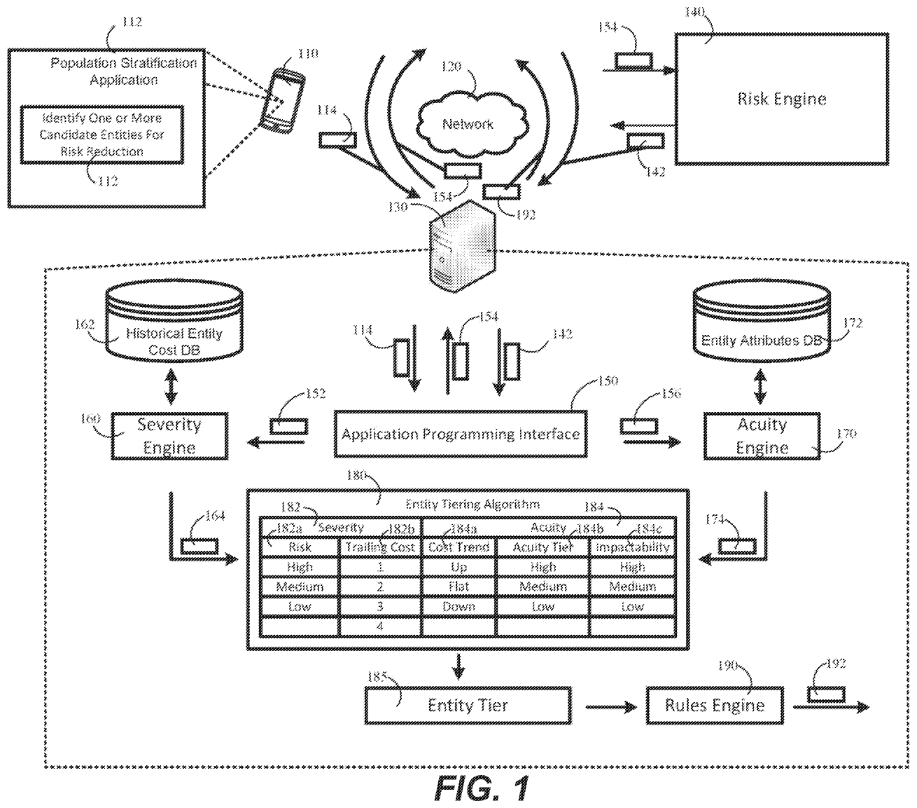

FIG. 1 is a contextual diagram of an example of a database management system 100 for dynamic population stratification based on data structures having fields structuring data related to changing entity attributes. The database management system may also be referred to as system 100.

The system 100 can include a user device 110, a network 120, an application server 130, and a risk engine 140. Though the risk engine 140 is depicted as being located remotely from the computer 130, the present disclosure is not so limited. Instead, of being hosted by a remote computer, and made available to the application server 130 using one or more networks 120, the risk engine 140 may also be stored and executed by the application server 130. The network 120 can include a wired Ethernet network, a wireless network, an optical network, a WAN, a LAN, a cellular network, the Internet, or any combination thereof.

In some implementations, a process executed by the system 100 can be initiated in response to a command from the user device 110. For example, the user device 110 can display a user interface 112 of an application such as a population stratification application. The application can include native application that include software instructions stored and executed on the user device 110. Alternatively, the application can include a web application that provides interface and controls for display in a web browser implemented by the user device 110. The user interface 112 can include one or more interactive controls that allows a user of the user device to instruct the user device 110 to initiate a process for identifying one or more candidate entities for risk reduction.

Responsive to the instruction from the user, the user device can transmit the instruction 114 to the application server 130 using the network 120. The instruction 114 can instruct the application server 130 to initiate the process for identifying one or more candidate entities for risk reduction. In some implementations, the instruction 114 can identify a particular entity and initiate performance a targeted process that generates an initial tier for the entity and applies one or more rules to data describing the entity to determine one or more strategies for moving the entity to a lower risk tier. In other implementations, the instruction 114 can be more general, and instead, request that the application server analyze a population of one or more entities. In some implementations, a user of the user device 110 can select a particular population of users for analysis such as populations of user associated with a particular geographic region, a particular genetic background, a particular ethnicity. Alternatively, or in addition, any other attribute can be used to define a population of user.

The application programming interface 150 can receive the instruction 114, and generate a request 154 for a risk score from the risk engine 140 for one or more entities. The application programming interface 150 can include software, hardware, or any combination thereof that functions as middleware between the components external to the application server 130 such as the population stratification application and the risk engine and components internal to the application server 130 such as the severity engine 160 and the acuity engine 170. In some implementations, the request 154 may include fields of a data structure structuring data attributes of a particular entity. In some implementations, the data attributes may include an entity identifier, historical health records associated with the entity, other attributes of the entity, or any combination thereof. In some implementations, the health records may be segmented into behavioral health records and physical health records. In other implementations, the request 154 may include fields of a data structure structuring data identifying a population of entities. In some implementations, the request 154 can include a stream of data describing various data attributes of each entity in the population. In other implementations, the request 154 may merely identify the population of entity and a computer hosting the risk engine an obtain information describing each respective entity of the population for input to the risk engine, which can process the input information generate a risk score for each entity. The application programming interface can transmit the request 154 to the risk engine using the network 120.

A computer hosting the risk engine 140 can receive the message 154 and obtain information about the one or more entities identified by the message 154 for inputs to the risk engine 140. In some implementations, this information may be obtained directly from the message 154, or stream of information associated therewith. In other implementations, the information may be obtained from an entity database accessible by the computer hosting the risk engine 140 that includes entity data. In some implementations, the inputs to the risk engine may include data describing behavioral health of an entity and physical health of the entity. The risk engine 140 can include one or more machine learning models that have been trained to predict a physical health risk associated with an entity, a behavioral risk associated with an entity, and the total cost associated with the entity. The risk engine 140 can generate a physical health risk associated with each entity identified by the message 154, a behavioral risk associated each entity identified by the message 154, and a total health cost associated with each entity identified by the message 154. Entities identified by the message 154 can include any entity identified by the message 154, or an accompanying data stream. In some implementations, only a single entity can be identified by the message 154. Alternatively, a population of entities can be identified by message 154.

The output 142 of the risk engine 140 can include data describing a level of risk associated with the entity whose data attributes were input to the risk engine 140. In some implementations, the risk may include a health risk. In some implementations, the risk may include a risk that the entity will result in high insurance costs. In some implementations, output 142 of the risk engine can be described as representing a medical complexity of an entity in a population and provide a prediction of future cost for the entity. In some implementations, the output 142, for each entity identified by the message 154, can include three separate risk scores that include a physical health risk, a behavioral health risk, and a total health risk. In some implementations, each respective risk score may be described in terms of a cost for each respective type of risk. Behavioral health risks can include conditions such as alcohol abuse and physical health risks can include congestive heart failure.

In some implementations, the risk engine 140 is configured to obtain a health profile for each entity identified by the message 154. The risk engine can map the health profile for each entity identified by the message 154 to categories defined by the Chronic Illness and Disability Payment System (CDPS), or other classification system, to arrive at a physical health risk score. By way of example, the risk engine 140 can map an entity's physical health related diagnosis and drug history described by the health profile can be mapped to the CDPS to arrive at the physical health risk score. The CDPS can include a diagnostic classification system for use in making health-based payments.

In some implementations, the risk engine 140 can use a behavioral health grouping function to map the health profile of each entity identified by the message 154 to behavioral health categories. By way of example, behavioral health related diagnoses and drug history described by the health profile can be mapped to one or more a behavioral health groups. In some implementation, there may exist more behavioral health group categories than CDPS categories. By way of example, the behavioral health groups can include 51 distinct categories, which is in sharp contrast to the original 6 CDPS categories. The additional behavioral health categories can provide more granularity and specificity to the behavioral risk model relative to the smaller number of CDPS categories. The total risk score is a combination score that is based on the physical health risks and behavioral health risks.

The risk engine 140 can perform predictive modeling once each entity's health profile is mapped into physical health categories such as CDPS categories and behavioral health categories. The predictive modeling can be implemented using one or more machine learning models trained to determine one or more risk scores for each entity such as a physical health risk score, a behavioral health risk score, and a total health risk score. In some implementations, the predicted risk score can be indicative of an entity's annual estimated medical costs. Once the risk scores, predicted costs, or both, are obtained for each entity, the risk engine 140 can rank the members based on their risk score, predicted costs, or both. In some implementations, the ranking is performed using percentile estimates such that an entity having the largest predicted cost would be at 100 percentile and an entity with smallest predicted loss would be at 0 percentile. Such a ranking may be performed whether the risk engine was used to generate risk scores, costs, or both, for single entity or a population of entities. For example, in implementations where only a single entity's risk score, costs, or both, where determined by the risk engine 140, a percentile rank can be determined for the entity based on known rankings of other entities stored by the risk engine 140 or other computing system. In some implementations, separate rankings can be determined for physical health risks, behavioral health risks, total health risks, costs associated with each type of risk, respectively. The training of the risk engine 140, use of the trained risk engine to determine one or more risk scores, ranking of entities, and other features of the risk engine 140 are described in more detail in US Pat. Publication 2019/0057320, which is hereby incorporated by reference in its entirety.

The data 142 output by the risk engine 140 can be provided to the application server 130. The data 142 output by the risk engine 140 can be received by the application programming interface 150. The application programming interface 150 can generate input data 152 to the severity engine 160 based on the data 142 received from the risk engine 140. In some implementations, the input data 152 can include one or more one or more entity identifiers and one or more risk scores received from the risk engine 140 and that correspond the one or more respective entities.

The severity engine 160 can be configured to determine one or more severity parameters that are to be used by the entity tiering algorithm to determine a tier for an entity. The severity engine 160 determines the one or more severity parameters based on the input data 152 received from the application programming interface 150. The input data 152 received by the severity engine 160 can include the one or more risk scores for the risk engine 140 and the entity identifier that corresponds to the risk scores. In some implementations, the severity engine 160 can access a historical entity cost database 162 to obtain historical cost data associated with the entity identified by the input data 152. Historical entity cost, for an entity, that is obtained from the historical entity cost database 162 can include, for example, a trailing healthcare related cost for the entity associated with the risk scores and identified by the input data 152. In some implementations, the trailing cost can include a 12-month trailing cost for the entity associated with the risk scores and identified by input data 152. The trailing 12-month cost of the entity may include the entity's healthcare related cost over the preceding 12 month time period. In some implementations, the severity engine may translate the historical costs of healthcare related services for the entity into a representative value that can be used as a proxy for an entity's trailing 12-month cost. For example, the severity engine can map 12-month trailing healthcare costs to a proxy value such as 1, 2, 3, or 4 with 1 representing the highest trailing costs and 1 representing the lowest trailing cost. Though an example of four proxy values is provided herein, more or less proxy values may be used to represent 12-month trailing cost values.

The severity engine 160 can provide an input 164 to the entity tiering algorithm 180 based on the risk scores obtained in the input data 152 and the historical entity costs obtained from the historical entity cost database 162. In some implementations, the respective risk values and historical costs may be provided to the entity tiering algorithm 180 as multiple separate parameters. For example, one or more risk scores and a trailing 12-month cost of healthcare related cost for the entity may be provide as inputs to the entity tiering algorithm 180. In other implementations, the respective risk values and historical costs may be provided to the entity tiering algorithm as a single parameter that is generated based on the combination of the risk scores and the historical costs. For example, one or more risk scores, or an average thereof, may be used as a multiplier for a trailing 12-month healthcare related costs for the entity to generate a single parameter for input to the entity tiering algorithm. In some implementations, the severity parameters 164 for an entity may be referred to as a ranking for the entity.

The inputs 164 provide parameters to the entity tiering algorithm 180 that enable the entity tiering algorithm to weigh the severity of the entity's health risks. By way of example, these inputs 164 that include data representing one or more risk scores and historical healthcare related costs enable the entity tiering algorithm to determine a degree of complexity of medical and behavioral health needs of the entity associated with the inputs 164. In some implementations, the tiering algorithm 180 can also determine, based on the inputs 164, if further information is required for the entity associated with the inputs 164. For example, the entity tiering algorithm can be programmed to generate one or more notification flags in the application server 130 for one or more entities identified as being a high risk entity (e.g., High) with very low trailing cost (e.g., 1). Such entities could be very well-managed, could be an entity who is not getting the appropriate level of care given their medical condition, or other factors which may require further consideration. The application server 130 can generate or notify a user of the user device 110 of such entity's so that treatment of these outlier entity's can be individually tailored.

Before, or after, the receipt of the output data 142 from the risk engine, the application programming interface 150 can provide input data 156 to the acuity engine 170. The input data 156 provided to the acuity engine 170 can include data identifying one or more entities. The one or more entities can include a single entity or a population of entities. The acuity engine 170 is configured to generate multiple types of data that when analyzed, collectively by the entity tiering algorithm 180, enable the entity tiering algorithm 180 to consider the acuity of the entity's condition when determining an initial tier for the entity. The multiple types of data include a measure of recent cost trend 184a, a measure of an acuity tier 184b, and a measure of impactability 184c.

The measure of recent cost trend 184a can include data describing whether an entity's recent healthcare related costs are trending up, trending down, or remaining flat. In some implementations, the measure of recent cost trend may only include data describing the entity's recent incursion of healthcare related costs related to high-intensity services. Recent healthcare related costs may include healthcare related costs accrued within a relatively recent period of time including a most recent two weeks, a most recent month, a most recent 6 weeks, a most recent 2 months, a most recent 10 weeks, a most recent 3 months, or the like. The measure of recent contest trend can be weighed, by the entity tiering algorithm 180, to identify entity's whose use of healthcare related services is increasing relative to their prior year. In some implementations, the entity's prior year healthcare costs can be representing using the trailing 12-year healthcare related costs for the entity. The acuity engine 184a can obtain data describing the entity's recent healthcare related costs from the entity attributes database 172.

The measure of an acuity tier 184b can include data representing an experience-based rating of recent entity acuity based on the entity's utilization of healthcare related services in the recent past. In some implementations, the utilized healthcare related services may include high-intensity services. High-intensity services can include recent admissions to a hospital, recent admissions to an emergency room, recent reporting, discovery, or diagnosis of chronic conditions, recent reporting, discovery, or diagnosis of unique medications for treatment of chronic conditions, recent utilization of outpatient primary care physician (PCP) or treating specialist types by unique TIN. The acuity engine 180 can determine information describing the entity's recent utilization of healthcare related services, including the entity's use of high-intensity related services, by accessing and retrieving entity attribute data from the entity attributes database 172. A use of a healthcare related service may be treated as “recent” if the use of the healthcare related service occurred within the past 90 days.

The acuity engine 180 can assign points to the entity for each of the following occurrences. For example, the acuity engine 180 can assign 1 point per number of in person (IP) hospital admissions greater than or equal to 2 two visits in the past 90 days. By way of another example, the acuity engine 180 can assign 1 point per number of emergency room (ER) visits greater than or equal to 2 in past 90 days. By way of another example, the acuity engine 180 can assign 1 point per number of chronic conditions reported, discovered, or diagnosed that are greater than or equal to 4 in past 12 months. By way of another example, the acuity engine 180 can assign 1 point for each unique medication for treatment of chronic conditions greater than or equal to 9 in 12 months. By way of another example, the acuity engine 180 can assign 1 point for each outpatient primary care physician (PCP) or treating specialist types by unique TIN greater than or equal to 10 in past 12 months.

The acuity engine 180 can perform one or more mathematical operations on the assigned points and determine an acuity score. By way of example, the acuity engine 180 can add assigned points and then determine an acuity score based on the sum of the points. In one implementation, for example, the acuity engine 180 can map the sum of the assigned points to a predetermined scale that defines a measure of acuity using one or more categories. For example, in some implementations, the categories of acuity may be defined as Low, Medium, or High using a scale of 0 to 5. In such an implementations, an acuity score ranking from 0 to 1 can be considered “Low” acuity, an acuity score ranging from 2 to 3 can be considered “Medium” acuity, and an acuity score ranging from 4 to 5 can be considered “High” acuity.

The measure of an impact score 184c to can be used to timely identify members whose pattern of care has recently shifted. The acuity engine 180 can generate the impact score 184 using an algorithm that analyzes the impactability related to an entities healthcare based on historical and present care pathway markers. The impact score can be assigned to each entity based on several factors including utilization trends, avoidable hospital visits, avoidable ER visits, missed outpatient follow-ups, or PCP visits. The acuity engine 180 can determine an impact score based on an evaluation of a number of different factors that includes potentially avoidable clinical events (e.g., avoidable ED visits, ambulatory Sensitive Conditions, or the like), behavioral health risk score determined by the risk engine, frequent use incidents, readmissions, hospitalization or ED visit without follow-up, trends in provider and medication use, medication adherence, and information obtained via health risk assessment (e.g., housing instability, financial instability, safety, social supports, or the like). The acuity engine 180 can determine an impact score based on the aforementioned factors and map the impact score to one or more categories. In some implementations, the categories may include a “High” impact score category, a “Medium” impact score category, and a “Low” impact score category. A “High” impact score can indicate a high shift, or high deviation, in the entity's recent pattern of care where the entity has recently been utilizing more healthcare services than historically used by the entity. A “Low” impact score can indicate a low shift, or low deviation, in the entity's recent pattern of care where the entity has recently been utilizing less healthcare services than historically used by the entity. A “Moderate” impact score can indicate that the entity's recent pattern of care has maintained relatively flat when compared to historically usage of healthcare service.

The aforementioned acuity data such as the measure of recent cost trend 184a, the measure of acuity tier 184b, and the measure of impactability 184c, can be analyzed by the entity tiering algorithm 180 and used, by the entity tiering algorithm 180, or one or more modules analyzing the output of the entity tiering algorithm 180, to identify new entities to a healthcare plan whose claim history is insufficient to score high on either risk or trailing cost. In some implementations, acuity factors such as use of medications for chronic conditions or ED utilization in a Low Risk member can be sought by application of one or more rules by the rules engine as a possible marker to flag a new member who needs additional support right away. The acuity engine 170 can provide the generate acuity data such as the measure of recent cost trend 184a, the measure of acuity tier 184b, and the measure of impactability 184c as input parameters 174 to the entity tiering algorithm. In some implementations, the acuity parameters 174 for an entity may be described as a second ranking.

The entity tiering algorithm 180 can analyze the severity input parameters 164 and the acuity input parameters 174 received from the severity engine 160 and acuity engine 170. The entity tiering algorithm 180 can determine a particular entity tier 185, of multiple entity tiers, to which the entity is to be initially assigned based on the severity input parameters 164 and the acuity input parameters 174. Each entity tier may include a particular category of entities associated with a health risk level defined by the particular category. In some implementations, the multiple entity tiers may include four tiers that rank from 1, indicating of a highest risk associate with an entity, to 4, indicating a lowest risk associated with the entity.

The entity tiering algorithm 180 view the severity and acuity parameters, collectively, to determine an extent that recent changes in entity attributes described by the entity's acuity parameters should change the overall assigned tier 185 of the entity, based on the acuity parameters highlighting a change in the recent trends of healthcare usage by the entity. Such trends may be increased in healthcare services usage, decreases in healthcare services uses, or flat healthcare services use (e.g., the entity's healthcare services usage has not substantially increased or decreased recently).

In some implementations, the entity tiering algorithm 180 can use a mathematical approach. In such an approach, data structures having fields that structure data representing each of the severity input parameters 164 and the acuity input parameters 174 can be accessed, and the data structured by the fields of the data structure and that represent each of the severity input parameters 164 and the acuity input parameters 174 can be mapped to respective numerical values. Then, the entity tiering algorithm 180 can determine, based on the respective numerical values, a particular entity tier 185 to which the entity should be assigned. In some implementations, the entity tiering algorithm 180 can generate a weight sum of the numerically translated severity input parameters 164 and the numerically translated acuity input parameters 174. In such implementations, the entity tiering algorithm 180 can determine, based on the weighted sum, a particular entity tier 185 to which the entity is to be assigned. In some implementations, each entity tier 185 of multiple entity tiers may be defined by a range of numerical values to which the weighted sum may be compared. In such implementations, the entity can be assigned to the entity tier if the weighted sum for the entity's numerically translated severity input parameters and numerically translated acuity input parameters falls within the range for the entity tier 185.

In other implementations, the entity tiering algorithm 180 can include a rules-based approach that evaluates the particular combination severity input parameters 164 and the acuity input parameters 174 provided as input parameters 180 to determine a particular entity tier 185 to which the entity is to be assigned. The rules-based approach can include an application of one or more rules that test for the occurrence of particular combinations of severity input parameters 164 and acuity input parameters 174. In some implementations, each particular entity tier 185 of the multiple entity tiers may be directly associated with one or more particular combinations of severity input parameters 164 and one or more acuity input parameters 174. By way of example, in some implementations, the entity tiering algorithm 180 can have a direct mapping of the severity input parameter 164 values of “Medium” risk, “Medium-Low” trailing cost or “2”, a “Flat” Cost Trend, a “Low” Acuity Tier, and a “Low” Impactability corresponds to an assigned entity tier 185 of Tier 3 based on the direct mapping of severity input parameters 164 and acuity input parameters 174 to the set of severity input parameters and acuity input parameters that define Tier 3. Each of the other tiers of the multiple tiers may have one or more corresponding sets of severity input parameters and acuity input parameters that define the tier.

By way of example, the entity tiering algorithm can determine, based on an analysis of severity 182 parameters and acuity 184 parameters associated with the entity, that the entity's has a High Risk 182a, High Trailing Cost 182b, Upward Recent Cost Trend 184a, High Acuity 184b, and High Impactability 184c (H,H,Up,H,H). The tiering algorithm may assign this entity to an entity tier 185 of “1” because this member is relatively complex from a medical perspective. For instance, for this entity in this example, the entity tiering algorithm as determined that the entity has accessed a high level of services over the last year, has very recently had a further increase in acuity, with a jump in cost, use of high levels of care, and the presence of factors that are potentially modifiable. Accordingly, the entity tiering algorithm would classify this entity as a Tier 1 rank, thereby associating the entity with a highest risk level tier.

By way of another example, the entity tiering algorithm 180 can determine, based on an analysis of other severity and acuity parameters for a different entity, that the different entity is associated with Low Risk 182a, Low Trailing Cost 182b, Upward Recent Cost Trend 184a, High Acuity 184b, and High Impactability 184c (L,L,Up,H,H). The tiering algorithm may assign his entity to an entity tier 185 of “2” because this entity has been relatively healthy (low risk). For example, this entity has not accessed services over the last year, but has very recently had an increase in acuity, with a jump in cost, use of high levels of care, and an increase in factors that are potentially modifiable. Accordingly, the entity tiering algorithm would classify this entity as a Tier 2 rank, thereby associated the entity with a lower level of risk than an entity classified in the highest risk level of Tier 1.

While these examples provide an example of the classification of respective entities into respective entity tiers, the present disclosure need not be so limited. Instead, the tiering algorithm could be configured to map entities having certain tiers to a desired Tier using custom rules and weighting of the algorithm that can be configured to weigh each of the severity and acuity data types accordingly.

In some implementations, the entity tiering algorithm 180 can process severity parameters 182 and acuity parameters 183 for each entity of a population of entities. Thus, while the entity tier 185 can be used to represent the particular tier that a particular entity was classified into, the entity tier 185 is not so limited. Instead, the entity tier 185 can be used to represent the entity tier that each respective entity of the population of entities was classified into. In some implementations, the tiers are of descending size and medical need with a first tier such as Tier 1 being the smallest tier and representing members with the highest medical need and a second tier such as Tier 4 containing the majority of members who have the lowest medical need. Tiers 2 and Tier 3 can represent those members that fall between Tiers 1 and 4 based, and classification of entities into such tiers will be based on whether their respective severity and acuity parameters make the respective entities more similar to Tier 1 or Tier 4, respectively, with Tier 2 being closer in similar to Tier 1 and Tier 3 being closer in similar to Tier 4. Within the higher level population tier, members are further ranked in descending order by their respective impact scores. The resulting raking structure can then be analyzed in order to prioritize and allocate entities based on their rank.

In some implementations, the entity tiering algorithm 180 can generate data for display, and provide the generated data to the user device 110 using the network 120, that shows the table of potential severity 182 and acuity 184 parameter values on the display of the user device 110. The generated data, when rendered by the user device, can highlight using one or more colors a particular value in each column that corresponds to the severity input parameters 164 and the acuity input parameters 174 of the entity under analysis. For example, in some implementations, if an entity is associated with parameters of “Medium” risk, “2” trailing cost, “UP” cost trend, “Medium” Acuity, and “High” impactability, then the application server 130 can generate data that, when rendered by the user device, highlights the cells of the table of severity 182 and acuity 184 parameters that correspond to “Medium” risk, “2” trailing cost, “UP” cost trend, “Medium” Acuity, and “High” impactability using a particular color such as red.

One the one or more entities have been assigned to a particular entity tier 185, the rules engine can apply one or more business rules to one or more entities assigned to a particular entity tier 185 to determine a cost associated with moving one or more entities to a different entity tier. For example, assume that an entity was assigned to an initial Tier “1” indicating that the entity is a high risk, high cost medical patient. One or more of the rules 190 can be applied to the entity's health data such as the entity's health profile, the entity's severity and acuity input parameters 164, 174 respectively, or a combination of both, to determine a cost of moving the entity from Tier “1” to a lower category of risk such as Tier “2” representing a medium-high category of risk.

Based on the application of the rules 190, the application server 130 can identify one or more of the severity 182 or acuity 184 data types that, if modified, would result in the entity being associated with the lower risk category. In some implementations, the application server 130 can generate data 192 that, when rendered by the user device 110, causes the user device to highlight cells of the table of severity 182 and acuity 184 data types displayed on the user device 110 using a different color such as blue. In such implementations, a message can be output to accommodate the change in color of the cells to communicate that if the entity associated with cells in the first color such as red is put into one or more programs to change the entity's severity an acuity parameters to those highlighted in the different color such as blue, then the entity can be projected to move to the tier associated with the different colored highlighting. In some implementations, the application server 130 can generate a cost of moving implementing one or more programs, regimens, or the like to move the entity from the higher cost, higher risk Tier “1” to a lower cost, lower risk Tier “2” and provide the generated cost for output on the display on the user device 110.

In some implementations, the application server 130 can generate a determined cost for moving the entity to each lower cost tier available. For example, the application server 130 can generate a cost to move an entity assigned to Tier “1” to Tier “2”, “Tier 3”, and “Tier 4,” respectively. The application server 130 can generate a program that can be implemented to achieve the move of the entity from the higher cost Tier “1” to each of the respective lower tiers. Then, the different costs and programs can be provided for display on the user device 110.

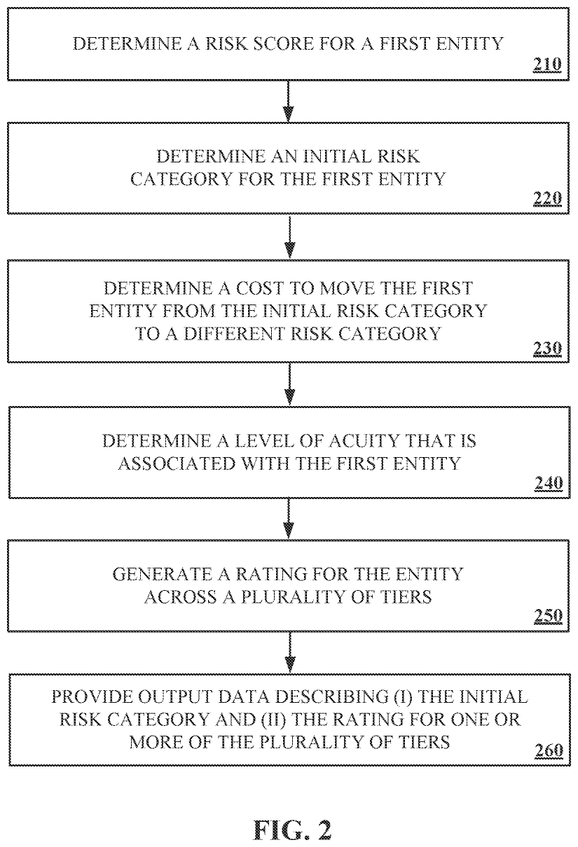

FIG. 2 is a flowchart of an example of a process 200 for dynamic population stratification based on changing entity attributes. In one implementation, the process 200 can include determining a risk score for a first entity (210), determining an initial risk category for the first entity based on an analysis of the determined risk score in view of a set of respective risk score for each second entity in a predetermined population of second entities (220), determining a cost to move the first entity from the initial risk category to a different risk category (230), determining a level of acuity that is associated with the first entity (240), generating a rating for the entity across a plurality of tiers based on the initial risk category, the different risk category, the determined cost, and the determined level of acuity (250), and providing output data describing (I) the initial risk category and (ii) the rating for one or more of the plurality of tiers (260).

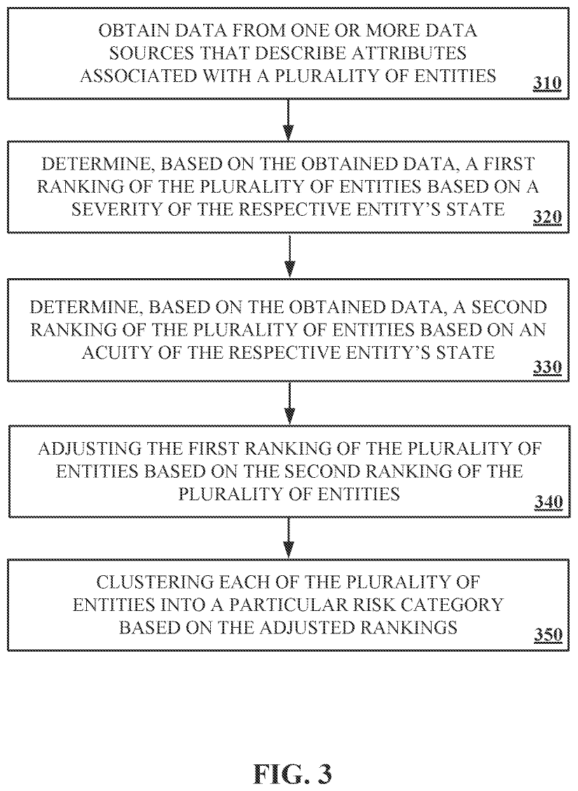

FIG. 3 is another flowchart of another example of a process 300 for dynamic population stratification based on changing entity attributes. In one implementations, the process 300 can include obtaining data from one or more data sources that describes attributes associated with a plurality of entities (310), determining, based on the obtained data, a first ranking of the plurality of entities, wherein the first ranking is based on a first measure of a severity state of each respective entity (320), determining, based on the obtained data, a second ranking of the plurality of entities, wherein the second ranking is based on a second measure of an acuity state of each respective entity (330), adjusting the first severity ranking of the plurality of entities based on the second acuity ranking of the plurality of entities (340), and clustering each of the plurality of entities to a particular risk category based on the adjusted rankings that is indicative of both the severity state and the acuity state of the respective entities (350).

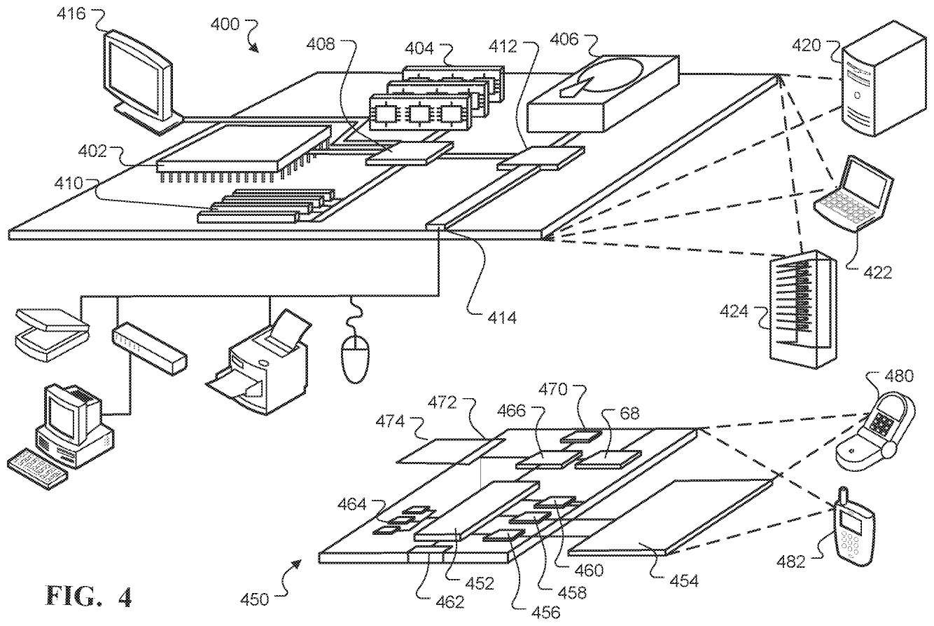

FIG. 4 is a block diagram of system components that can be used to implement a database management system for dynamic population stratification based on data structures having fields structuring data related to changing entity attributes.

Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smartphones, and other similar computing devices. Additionally, computing device 400 or 450 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 400 includes a processor 402, memory 404, a storage device 408, a high-speed interface 408 connecting to memory 404 and high-speed expansion ports 410, and a low speed interface 412 connecting to low speed bus 414 and storage device 408. Each of the components 402, 404, 408, 408, 410, and 412, are interconnected using various busses, and can be mounted on a common motherboard or in other manners as appropriate. The processor 402 can process instructions for execution within the computing device 400, including instructions stored in the memory 404 or on the storage device 408 to display graphical information for a GUI on an external input/output device, such as display 416 coupled to high speed interface 408. In other implementations, multiple processors and/or multiple buses can be used, as appropriate, along with multiple memories and types of memory. Also, multiple computing devices 400 can be connected, with each device providing portions of the necessary operations, e.g., as a server bank, a group of blade servers, or a multi-processor system.

The memory 404 stores information within the computing device 400. In one implementation, the memory 404 is a volatile memory unit or units. In another implementation, the memory 404 is a non-volatile memory unit or units. The memory 404 can also be another form of computer-readable medium, such as a magnetic or optical disk.

The storage device 408 is capable of providing mass storage for the computing device 400. In one implementation, the storage device 408 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 404, the storage device 408, or memory on processor 402.

The high speed controller 408 manages bandwidth-intensive operations for the computing device 400, while the low speed controller 412 manages lower bandwidth intensive operations. Such allocation of functions is exemplary only. In one implementation, the high-speed controller 408 is coupled to memory 404, display 416, e.g., through a graphics processor or accelerator, and to high-speed expansion ports 410, which can accept various expansion cards (not shown). In the implementation, low-speed controller 412 is coupled to storage device 408 and low-speed expansion port 414. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 400 can be implemented in a number of different forms, as shown in the figure. For example, it can be implemented as a standard server 420, or multiple times in a group of such servers. It can also be implemented as part of a rack server system 424. In addition, it can be implemented in a personal computer such as a laptop computer 422. Alternatively, components from computing device 400 can be combined with other components in a mobile device (not shown), such as device 450. Each of such devices can contain one or more of computing device 400, 450, and an entire system can be made up of multiple computing devices 400, 450 communicating with each other.

The computing device 400 can be implemented in a number of different forms, as shown in the figure. For example, it can be implemented as a standard server 420, or multiple times in a group of such servers. It can also be implemented as part of a rack server system 424. In addition, it can be implemented in a personal computer such as a laptop computer 422. Alternatively, components from computing device 400 can be combined with other components in a mobile device (not shown), such as device 450. Each of such devices can contain one or more of computing device 400, 450, and an entire system can be made up of multiple computing devices 400, 450 communicating with each other.

Computing device 450 includes a processor 452, memory 464, and an input/output device such as a display 454, a communication interface 466, and a transceiver 468, among other components. The device 450 can also be provided with a storage device, such as a micro-drive or other device, to provide additional storage. Each of the components 450, 452, 464, 454, 466, and 468, are interconnected using various buses, and several of the components can be mounted on a common motherboard or in other manners as appropriate.

The processor 452 can execute instructions within the computing device 450, including instructions stored in the memory 464. The processor can be implemented as a chipset of chips that include separate and multiple analog and digital processors. Additionally, the processor can be implemented using any of a number of architectures. For example, the processor 410 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 450, such as control of user interfaces, applications run by device 450, and wireless communication by device 450.

Processor 452 can communicate with a user through control interface 458 and display interface 456 coupled to a display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a user. The control interface 458 can receive commands from a user and convert them for submission to the processor 452. In addition, an external interface 462 can be provide in communication with processor 452, so as to enable near area communication of device 450 with other devices. External interface 462 can provide, for example, for wired communication in some implementations, or for wireless communication in other implementations, and multiple interfaces can also be used.

The memory 464 stores information within the computing device 450. The memory 464 can be implemented as one or more of a computer-readable medium or media, a volatile memory unit or units, or a non-volatile memory unit or units. Expansion memory 474 can also be provided and connected to device 450 through expansion interface 472, which can include, for example, a SIMM (Single In Line Memory Module) card interface. Such expansion memory 474 can provide extra storage space for device 450, or can also store applications or other information for device 450. Specifically, expansion memory 474 can include instructions to carry out or supplement the processes described above, and can include secure information also. Thus, for example, expansion memory 474 can be provide as a security module for device 450, and can be programmed with instructions that permit secure use of device 450. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 464, expansion memory 474, or memory on processor 452 that can be received, for example, over transceiver 468 or external interface 462.

Device 450 can communicate wirelessly through communication interface 466, which can include digital signal processing circuitry where necessary. Communication interface 466 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 468. In addition, short-range communication can occur, such as using a Bluetooth, Wi-Fi, or other such transceiver (not shown). In addition, GPS (Global Positioning System) receiver module 470 can provide additional navigation- and location-related wireless data to device 450, which can be used as appropriate by applications running on device 450.

Device 450 can also communicate audibly using audio codec 460, which can receive spoken information from a user and convert it to usable digital information. Audio codec 460 can likewise generate audible sound for a user, such as through a speaker, e.g., in a handset of device 450. Such sound can include sound from voice telephone calls, can include recorded sound, e.g., voice messages, music files, etc. and can also include sound generated by applications operating on device 450.

The computing device 450 can be implemented in a number of different forms, as shown in the figure. For example, it can be implemented as a cellular telephone 480. It can also be implemented as part of a smartphone 482, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1-15. (canceled)

16. A system, comprising:
a memory configured to store:
an entity profile comprising a plurality profile data items of an entity;
a predictive machine learning (ML) model trained on a training dataset comprising one or more physical health (PH) categories, one or more behavioral health (BH) categories, and historical medical costs to derive a coefficient for each of the one or more PH categories and BH categories as independent variables with respect to a historical medical cost, wherein the one or more PH categories and the one or more BH categories are each independent variables and the historical medical costs are each a dependent variable;
a processor programmed to:
map each profile data item from the entity profile to: (i) the one or more PH categories or (ii) the one or more BH categories;
generate, by the predictive ML model, a risk score and a predicted future estimated medical cost based on each of the mapped profile data items and the coefficients learned from the training dataset;
generate a multi-dimensional acuity of the entity that indicates a recent change in use of high intensity services used by the entity, the multi-dimensional acuity comprising an assessment of the recent change based on a plurality of dimensions;
assign the entity to a risk tier based on the risk score, the predicted future estimated medical cost, and the multi-dimensional acuity; and
evaluate the risk tier against one or more intervention assignment rules to identify a recommended intervention for the entity.

17. The system of claim 16, wherein the processor is further programmed to:
receive a data refresh that includes an update to the entity profile; and
refresh the risk tier assigned to the entity based on the updated entity profile.

18. The system of claim 17, wherein the processor is further programmed to:
retrain the predictive ML model to update the coefficients based on the data refresh.

19. The system of claim 16, wherein the training dataset comprises at least a first training dataset for adults and a second training dataset for children, wherein the derived coefficient from either is specifically used for the entity depending on whether the entity is a child or an adult.

20. The system of claim 16, wherein to generate the risk score, the processor is programmed to:
identify the one or more PH categories and the one or more BH categories to which the plurality profile data items have been mapped;
determine a PH risk score based on the identified one or more PH categories and a BH risk score based on the identified one or more BH categories; and
generate the risk score based on the PH risk score and the BH risk score.

21. The system of claim 16, wherein to generate the risk score, the processor is programmed to:
generate risk scores of other entities; and
rank the risk score with respect to the risk scores of the other entities to determine a relative percentile risk, wherein the assigned risk tier is based on the relative percentile risk.

22. The system of claim 16, wherein to assign the entity to a risk tier based on the risk score, the predicted future estimated medical cost, and the multi-dimensional acuity, the processor is programed to:
identify a path to the assigned risk tier, wherein the path represents a combination of individual values for the risk score, the predicted future estimated medical cost, and the multi-dimensional acuity that is associated with the assigned risk tier.

23. The system of claim 16, wherein to generate the multi-dimensional acuity, the processor is further programmed to:
identify a first cost for services used by the entity during a first time interval;
identify a second cost for services used by the entity during a second time interval longer than the first time interval; and
determine a cost trend based on the first cost and the second cost, wherein the multi-dimensional acuity is based on the cost trend.

24. The system of claim 16, wherein to generate the multi-dimensional acuity, the processor is further programmed to:
increment, for each of a plurality of services received by the entity within a time period, an acuity score; and
determine an acuity tier for the entity based on the acuity score, wherein the multi-dimensional acuity is based on the acuity tier.

25. The system of claim 16, wherein to generate the multi-dimensional acuity, the processor is further programmed to:
determine an impact score for the entity based on one or more historical pathway markers and/or one or more present care pathway markers, wherein the multi-dimensional acuity is based on the impact score.

26. A method, comprising:
storing, in a memory accessible to a processor:
an entity profile comprising a plurality profile data items of an entity;
a predictive machine learning (ML) model trained on a training dataset comprising one or more physical health (PH) categories, one or more behavioral health (BH) categories, and historical medical costs to derive a coefficient for each of the one or more PH categories and the one or more BH categories as independent variables with respect to a historical medical cost, wherein the one or more PH categories and the one or more BH categories are each independent variables and the historical medical costs are each a dependent variable;
mapping, by the processor, each profile data item from the entity profile to: (i) the one or more PH categories or (ii) the one or more BH categories;
generating, by the processor via the predictive ML model, a risk score and a predicted future estimated medical cost based on each of the mapped profile data items and the coefficients learned from the training dataset;
generating, by the processor, a multi-dimensional acuity of the entity that indicates a recent change in use of high intensity services used by the entity, the multi-dimensional acuity comprising an assessment of the recent change based on a plurality of dimensions;
assigning, by the processor, the entity to a risk tier based on the risk score, the predicted future estimated medical cost, and the multi-dimensional acuity; and
evaluating, by the processor, the risk tier against one or more intervention assignment rules to identify a recommended intervention for the entity.

27. The method of claim 26, the method further comprising:
receiving a data refresh that includes an update to the entity profile; and
refreshing the risk tier assigned to the entity based on the updated entity profile.

28. The method of claim 27, the method further comprising:
retraining the predictive ML model to update the coefficients based on the data refresh.

29. The method of claim 26, wherein the training dataset comprises at least a first training dataset for adults and a second training dataset for children, wherein the derived coefficient from either is specifically used for the entity depending on whether the entity is a child or an adult.

30. The method of claim 26, wherein generating the risk score comprises:
identifying the one or more PH categories and the one or more BH categories to which the plurality profile data items have been mapped;
determining a PH risk score based on the identified one or more PH categories and a BH risk score based on the identified one or more BH categories; and
generating the risk score based on the PH risk score and the BH risk score.

31. The method of claim 26, wherein generating the risk score comprises:
generating risk scores of other entities; and
ranking the risk score with respect to the risk scores of the other entities to determine a relative percentile risk, wherein the assigned risk tier is based on the relative percentile risk.

32. The method of claim 26, wherein assigning the entity to a risk tier based on the risk score, the predicted future estimated medical cost, and the multi-dimensional acuity comprises:
identify a path to the assigned risk tier, wherein the path represents a combination of individual values for the risk score, the predicted future estimated medical cost, and the multi-dimensional acuity that is associated with the assigned risk tier.

33. The method of claim 26, wherein generating the multi-dimensional acuity comprises:
identifying a first cost for services used by the entity during a first time interval;
identifying a second cost for services used by the entity during a second time interval longer than the first time interval; and
determining a cost trend based on the first cost and the second cost, wherein the multi-dimensional acuity is based on the cost trend.

34. The method of claim 26, wherein generating the multi-dimensional acuity comprises:
incrementing, for each of a plurality of services received by the entity within a time period, an acuity score; and
determining an acuity tier for the entity based on the acuity score, wherein the multi-dimensional acuity is based on the acuity tier.

35. A non-transitory computer readable medium storing instructions that, when executed by a processor, programs the processor to:
a processor programmed to:
map each profile data item from an entity profile to: (i) one or more physical health (PH) categories or (ii) one or more behavioral health (BH) categories, wherein the entity profile comprises a plurality profile data items of an entity;
generate, by a predictive machine learning (ML) model, a risk score and a predicted future estimated medical cost based on each of the mapped profile data items and coefficients learned from a training dataset;
wherein the predictive ML model is trained on the training dataset comprising the one or more PH categories, the one or more BH categories, and historical medical costs to derive a coefficient for each of the one or more PH categories and the one or more BH categories as independent variables with respect to a historical medical cost, wherein the one or more PH categories and the one or more BH categories are each independent variables and the historical medical costs are each a dependent variable;
generate a multi-dimensional acuity of the entity that indicates a recent change in use of high intensity services used by the entity, the multi-dimensional acuity comprising an assessment of the recent change based on a plurality of dimensions;
assign the entity to a risk tier based on the risk score, the predicted future estimated medical cost, and the multi-dimensional acuity; and
evaluate the risk tier against one or more intervention assignment rules to identify a recommended intervention for the entity.
